(19) United States
(12) Patent Application Publication
(10) Pub. No.: US 2026/0091526 A1
(43) Pub. Date: Apr. 2, 2026

(54) RAZOR CARTRIDGE

(71) Applicant: Harry's, Inc., New York, NY (US)

(72) Inventors: Molly DUDAS, Brooklyn, NY (US); Sarah ERDENBRECHER, Heldburg (DE); Alex CHENVAINU, Mahwah, NJ (US)

(21) Appl. No.: 19/341,078

(22) Filed: Sep. 26, 2025

Related U.S. Application Data
(60) Provisional application No. 63/700,045, filed on Sep. 27, 2024.

(51) Int. Cl. B26B 21/44; A61K 8/85; A61Q 9/02; B26B 21/40

(52) U.S. Cl. CPC B26B 21/443; A61K 8/85; A61Q 9/02; B26B 21/4018; B26B 21/4025; B26B 21/4068

(57) ABSTRACT

A razor cartridge comprising a core including a blade mounting portion configured to receive one or more blades and at least one of a portion of a frame, a guard and a cap, said razor cartridge comprising one or more skin contacting portions associated with one or more portions of the core, wherein one or more of the skin contacting portions comprise a lubricious layer formed of a lubricious layer composition including a matrix component and a lubricious component.

### FIELD OF THE INVENTION

The present invention relates to a wet shave razor cartridge and, more particularly, to a wet shave razor cartridge with a molded lubrication portion for reducing friction during shaving.

### BACKGROUND OF THE INVENTION

Razors with replaceable blade cartridges attached to non-disposable handles have become universally used by consumers for shaving. Such cartridges generally include a blade housing in which one or more shaving blades are mounted, a guard and a cap to help define the blade exposure, and means for connecting the blade housing to a handle.

It has become common to include a lubricious material on one or more of the skin contacting portions of the razor cartridge. The lubricious material often takes the form of a low-friction material used for a skin contacting portion of the cartridge or the application of a water-soluble lubricious material alone or incorporated into a polymer matrix on a skin contacting portion of the cartridge. One of the most common lubricious components on a razor cartridge is a lube strip placed in the area of the cap. Other such components may comprise a fin guard with a lubricious material adjacent the fin guard or on one or more areas adjacent to or surrounding the blade mounting portion of the cartridge.

In this regard, razor cartridges generally comprise one or more skin contacting portions in addition to the guard and cap which may comprise a lubricious material. These additional skin contacting portions generally include an area below the guard, an area above the cap and areas on the lateral sides of the blades, which may be referred to as the frame of the cartridge. More particularly, the frame generally surrounds the portion of the housing comprising the one or more blades to provide greater stability to the blades on the user's skin during shaving, thereby improving shaver comfort and making shaving easier by increasing the skin contact surface area of the area around the blades. This frame can be integral to the blade housing, as generally shown in U.S. Pat. No. 6,161,287, or attached to the blade housing, as generally shown in U.S. Pat. Nos. 6,185,823, 8,327,546, 9,434,080 and 9,517,570.

Along with the use of larger frames around the one or more blades to improve ease of shaving and user comfort, manufacturers have added low friction or lubricious materials onto the frames. In some instances, manufacturers have formed frames comprising low-friction materials and attached them to the blade housing. In other cases, manufacturers have applied lubricious layers onto the skin contacting portions of the razor cartridge, by coating, spraying, molding, etc., so that the lubricous material is made integral to the cartridge.

Existing lubricious material formulations generally include a high content of polystyrene, titanium dioxide or other substances that may be harmful to the user, and possibly dyes for defining the lubricious portion of the cartridge. However, these formulations often produce a lubricious material with a performance and feel described by customers as poor and unpleasant, where many customers find current formulations suffer from a feeling of cheap plastic.

Moreover, in instances where the lubricious material is formed as a layer on the razor cartridge by injection molding, heating and applying the lubricious material layer onto the underlying core creates physical challenges. In this regard, the injection molding process used for applying a lubricious material layer onto a razor cartridge is stressful for existing lubricous material resulting in degraded performance of the lubricating material. Moreover, the injection molding process for applying the lubricious material layer applies stress on the underlying core material due to thermal shrinkage, leading to overall warpage, which is undesired in the final product.

In addition, materials used to impart a lubricious characteristic, including lubricious materials on polystyrene matrices, have deficiencies based on the nature of the materials used and placement of the materials on the razor cartridge. Overcoming such deficiencies include the need to obtain a homogeneous surface with a simultaneous reduction of polystyrene material in the formulation, to improve the incorporation and distribution of dyes and pigments in large scale production of the lubricious material and the need for improving feel, specifically by reducing the feeling of plastic on the skin.

Known active polymers used to create a lubrication surface are also compromised by choosing the wrong matrix, which may result in unwanted interactions between the active polymers and other components. Moreover, adding the known lubricious materials as a layer to a razor cartridge has faced challenges, where applying a lubricious material to an underlying razor cartridge housing or frame may affect the underlying physical characteristics of the razor cartridge.

It is therefore an object of the invention to provide an improved lubricious material formulation and an improved cartridge structure, resulting in the production of an improved razor cartridge having a lubricious layer with improved feel and reduced physical deformation of the cartridge during manufacture.

### SUMMARY OF THE INVENTION

The present invention is directed to a razor cartridge comprising a core including a blade mounting portion configured to receive one or more blades and at least one of a portion of a frame, a guard and a cap, said razor cartridge comprising one or more skin contacting portions associated with one or more portions of the core, wherein one or more of the skin contacting portions comprise a lubricious layer formed of a lubricious layer composition including a matrix component and a lubricious component. In a preferred embodiment, the matrix component comprises a biodegradable copolymer, and most preferably polybutylene adipate terephthalate (PBAT).

The lubricious layer is preferably formed by extruding the material formulation to form an independent member that is then attached to one or more of the skin contacting areas associated with the core, injection molding the lubricious layer composition directly onto one or more of the skin contacting areas associated with the core, or a combination of these options.

For example, the lubricious layer can be formed as a lube strip by extruding the lubricious layer composition into a continuous independent strip, which is then cut into segments and attached to the core of the razor cartridge in the area of the cap. Alternatively, the lubricious layer material can be injection molded by overmolding the lubricious layer material directly onto the core, in a skin contacting portion associated with the core, including onto areas such as the cap, guard, or a portion of the frame that is part of the core, whether the frame is integral with the blade mounting portion or is attached to the blade mounting portion.

Moreover, different skin contacting portions of a razor cartridge can be formed of different lubricious layer compositions. For example, the lube strip can be formed from a composition with a lower amount of the matrix component and a higher amount of the lubricious component while the frame surrounding the blade mounting portion has a higher amount of the matrix component and a lower amount of the lubricious component.

The core of the razor cartridge is preferably formed of a suitable plastic, such as acrylonitrile butadiene styrene (ABS), as is well known in the art. The core is preferably injection molded as a unitary member including the blade mounting portion, the frame, and optionally one or more of the guard and cap. The lubricious layer is then attached to the core by affixing the independently formed lubricious layer onto a skin contacting portion associated with the core or by injection molding the lubricious layer onto a portion of the core associated with a skin contacting portion.

The lubricious layer can be any desired thickness, but it is preferred that the lubricious layer be from about 0.5 mm to about 5.0 mm, more preferably from about 0.75 mm to about 3.0 mm and most preferably from about 1.0 mm to about 2.0 mm. The thickness of the lubricious layer may also depend on the lubricious layer composition, where the thickness may be greater when using a material that degrades faster in water than when using a material that degrades slower, and/or on the thickness of the core member, where the thickness of the lubricious layer is preferably comparable to the thickness of the core member.

In a preferred embodiment, the core comprises the blade mounting portion and a frame that surrounds the blade mounting portion, where the frame is oversized to provide additional surface area for user comfort and ease when shaving. In this embodiment, the lubricious layer covers most all of the upper surface of the core in the area of the frame to further improve user comfort when shaving to maximize the ratio of lubrication surface area to skin contact surface area.

When the lubricious layer is applied by injection molding the molten lubricious layer material directly onto the core in a majority of the areas associated with the skin contacting portions, it is preferred that the lubrication layer and core are flush with one another to improve ejection of the part from the mold, reducing warpage of the parts during removal. Such cartridge warpage is particularly unwanted in a razor cartridge, where warping affects the shave plane created between the guard, cap and blades, resulting in potential nicks and cuts while shaving.

When the lubricious layer is overmolded directly onto the core in an area associated with one or more skin contacting portions, it is preferred that one or more of those areas are strengthened with support members. In this regard, the core preferably comprises a hollowed back profile to reduce the amount of plastic used to manufacture the core, which may otherwise be more susceptible to warping. In such an embodiment, the hollowed back preferably comprises one or more support members, such as ribs, to provide increased rigidity to the core. This is especially important where the core may also be subject to warping due to thermal shrinkage when the hot, molten lubricious layer is injection molded onto a larger portion of the core and then cools.

A preferred embodiment related to an overmolded lubricious layer further comprises interlocking features between the core and the lubricious layer to improve the physical connection between the core and the lubricious layer, which also reduces warpage and maintains a more precise shave plane. Preferred interlocking features may include openings that pass through the core, from the front surface to the back surface of the core, where the lubricious layer composition fills the openings when injection molded onto the core. Interlocking features of varying length aids in the flow of material along the varying curvature of the surface perimeter of the core, with the interlocking features preferably located on flat surfaces to improve ejection of the part.

At least some of the interior surfaces of the core to be covered by the lubricious layer are preferably curved or rounded to improve flow of the molten lubricious layer material over the core surfaces. For example, the transitions from side walls to bottom walls of cut outs to be overmolded with the lubricious layer may be rounded, in a convex or concave configuration, to improve flow.

Rounded surfaces on the perimeter of the core also improve ejection of the part, with a 5 to 10 degree exaggerated draft on outside surfaces, preferably about 7 degrees, also aiding in ejection, reducing warpage due to sticking in the mold tool. Extra core material on outside surfaces of the core, particularly forming a collar at the sides of the core adjacent the back edge, not only minimizes the lubricious layer composition material used, but also maximizes the surface area of the lubrication layer in the skin contacting portions of the razor cartridge.

Although the guard of the razor cartridge can be formed of a thermoplastic elastomer fin structure, as is well known in the art, a preferred embodiment of the present invention comprises a rigid guard made of the core material, preferably ABS. Such a plastic guard may be formed of a single or multiple rigid fins, including one or more solid longitudinal fins running parallel with the blades and/or one or more fins with breaks along the longitudinal length, breaking the fins up into two or more longitudinal sections.

The matrix component of the lubricious layer composition may comprise PBAT, which is a biodegradable copolymer, and particularly a copolyester that consists of aromatic and aliphatic components, where the monomers are adipic acid, 1,4-butanediol and terephthalic acid. PBAT is produced by a number of different manufacturers under brand names such as ECOFLEX® by BASF, WANGO by Zhuhai Wango Chemical Co Ltd, ECOWORLD® by JinHui ZhaoLong High Technology, EASTAR BIO by Eastman Chemical, and ORIGO-BI by Novamont. Variations include PBAT in a blend with poly(lactic acid) manufactured by BASF under the brand name ECOVIO® and PBAT in a blend with starch manufactured by Novamont under the brand name MATER-BI and by JinHui Zhao Long High Technology under the brand name ECOWILL.

The use of PBAT as an ingredient of the matrix component is particularly well suited to use in a lubricous layer composition due to its high flexibility and biodegradable nature. In addition to the environmentally friendly nature of the material, use of PBAT improves the smoothness of the surface and provides surfaces that are softer than normal. It also acts as a solubilizer/emulsifier, aiding in mixing the lubricious layer composition materials and deriving a homogenous color. Notwithstanding, PBAT has not yet been used for cosmetic products, in razors or in lubricious components of razors.

In a preferred embodiment, the PBAT comprises 2 to 25% of the lubricious layer composition, preferably 4 to 20% and most preferably 5-15%. Other preferred ingredients of the lubricious layer composition may include a thickener, such as the polyethylene oxide (PEO) thickener POLYOX, soothing agents such as aloe vera, colorants, antioxidants such as tocopherol and one or more resins, such as polystyrene as a portion of the matrix material.

The lubricious layer composition strategically balances polystyrene and PBAT, which allows the material to retain its structural integrity while minimizing the typical “plastic-like” feel associated with synthetic polymers. This blend improves both the tactile sensation and environmental profile of the material, combining durability with a more natural feel.

A preferred formulation for the lubricious layer composition comprises the following components, including the general purpose of each:
PEG115M (Polyethylene Glycol 115M): in an amount of about 60 to about 75 weight percent, preferably about 65 to about 70 weight percent, and most preferably about 68 weight percent. This component contributes to the viscosity and glide properties of the formulation.
Polystyrene: in an amount of about 10 to about 30 weight percent, preferably about 15 to about 25 weight percent and most preferably about 20 weight percent. Polystyrene offers structural integrity but is used in an optimized quantity to minimize the “plastic-like” feel.
PBAT (Polybutylene Adipate Terephthalate): in an amount of about 3 to about 13 weight percent, most preferably from about 6 to about 10 weight percent and most preferably about 8 weight percent. This biodegradable polyester provides flexibility and improves the performance of the formulation. It ensures an appealing look and a significantly improved feel of the product. The surface of plates feels much softer and therefore hugs the skin better.
The PBAT material can be added as a granulate to the remaining granulated materials directly before injection molding (granulate processing) or can be ground beforehand. If the materials are all ground, all components can be compounded together to obtain a granulate ready for injection molding. However, grinding the materials is very demanding and it is preferred that grinding be carried out using cryogenic grinding.
PEG90 (Polyethylene Glycol 90): in an amount of about 1 to about 7 weight percent, preferably in an amount of about 3 to about 5 weight percent, and most preferably about 4.0 weight percent. PEG90 supports the uniform distribution of dyes and increases glide performance.
Tocopherol: in an amount of about 0.01 to about 0.2 weight percent and preferably about 0.05 to about 0.15 weight percent and most preferably about 0.1 weight percent. Tocopherol, or Vitamin E, acts as an antioxidant and adds extra care properties.
Dye: in an amount of about 0.1 to about 0.4 weight percent, preferably about 0.2 to about 0.3 weight percent, and most preferably about 0.25 weight percent. The dye provides the desired color of the lubricious layer.
Care Agent: in an amount of about 0.001 to about 0.04, preferably about 0.01 to about 0.03 weight percent, and most preferably about 0.02 weight percent. Care agents enhance skin compatibility and offer additional benefits during use.

The use of polyethylene glycol components in the lubricious layer composition enables a homogeneous mixture of the dyes, preventing separation and clumping, where the uniform distribution of the dye results in an improved appearance of the lubricious layer. The combination of PEG115M and PEG90 provides excellent glide properties, reducing friction, and controlling the polystyrene content provides structural benefits without imparting an unpleasant “plastic-like” sensation from the lubricious layer. Tocopherol and other care agents further contribute to a pleasant skin feel and prevent irritation.

More to the point, the preferred formulation of the lubricious layer composition provides an optimized distribution of dyes and care components, an improved tactile experience with a reduced plastic-like sensation, and a biodegradable aspect, due to the use of PBAT, to reduce the environmental impact.

The formulation process preferably comprises a precise mixing of PEG115M, polystyrene, PBAT, PEG90, tocopherol, dyes, and care agents. Each specific ingredient is added in a specific order and under controlled conditions to ensure optimal blending and homogeneity, with the PBAT being ground using cryogenic grinding prior to blending. The blended material can be easily extruded or molded, making it versatile for different manufacturing processes.

### BRIEF DESCRIPTION OF THE DRAWINGS

The attached drawings, in which like reference characters represent like parts, are intended to better illustrate a preferred embodiment of the present invention without limiting the invention in any manner whatsoever.

FIG. 1 is a front elevation of the face of the razor cartridge of the present invention.

FIG. 2 is a perspective view of the embodiment of FIG. 1 with engineering lines.

FIG. 3A is a front elevation of an embodiment of the invention with lubricious layer extending in an arch to the guard area.

FIG. 3B is a front elevation of an embodiment of the invention with lubricious layer extending in a rectangular shape to the guard area.

FIG. 3C is a front elevation of an embodiment of the invention with lubricious layer extending in a curved shape into to guard area.

FIG. 3D is a front elevation of an embodiment of the invention with lubricious layer extending in a rectangular shape with rounded corners to the guard area.

FIG. 4 is a cross-sectional side elevation of an embodiment of the present invention through a center line.

FIG. 5 is a rear elevation of an embodiment of the present invention.

FIG. 6A is a perspective view of a preferred core, exploded from an overmolded lubricious layer (shown in FIG. 6B).

FIG. 6B is a perspective view of a preferred lubricious layer, exploded from an underlying core (shown in FIG. 6A).

FIG. 7 is a partial cross-sectional side elevation of an embodiment of the present invention at the cap of a razor cartridge.

FIG. 8 is a partial rear elevation of an embodiment of the present invention highlighting rounded surfaces on the rear of the core.

FIG. 9 is a partial rear elevation showing the lubricous layer extending through the core forming interlocking features.

FIG. 10 is a partial perspective view of the guard fins of an embodiment of the present invention.

FIG. 11 is a partial cross-sectional side elevation of the guard fins of an embodiment of the present invention.

FIG. 12A is a front elevation of an embodiment of the invention with a single longitudinal guard fin being formed integrally as part of the core.

FIG. 12B is a front elevation of an embodiment of the invention with a single longitudinal guard fin being formed integrally as part of the core in two longitudinal sections.

FIG. 12C is a front elevation of an embodiment of the invention with a single longitudinal guard fin being formed integrally as part of the core in three longitudinal sections.

FIG. 12D is a front elevation of an embodiment of the invention with a single longitudinal guard fin being formed integrally as part of the core in four longitudinal sections.

FIG. 12E is a front elevation of an embodiment of the invention with a single longitudinal guard fin being formed integrally as part of the core in five longitudinal sections.

FIG. 12F is a front elevation of an embodiment of the invention with a single longitudinal guard fin being formed integrally as part of the core in six longitudinal sections.

FIG. 13 is a perspective view of an embodiment of the invention with a comb portion in front of the longitudinal guard fin, both formed integrally as part of the core.

FIG. 14A is a cross-sectional side elevation of an embodiment of the invention with an independent lube strip inserted into an opening on the face of the cartridge core.

FIG. 14B is a cross-sectional side elevation of an embodiment of the invention showing the portion of the core having an opening in which an independent lube strip can be inserted on the face of the cartridge core.

FIG. 15 is a partial front elevation of an embodiment of the invention with an independent cap element on the face of the cartridge core.

### DETAILED DESCRIPTION OF THE PREFERRED EMBODIMENT

The following description of the preferred embodiment is presented to describe the present invention without limiting the scope of the invention in any manner whatsoever.

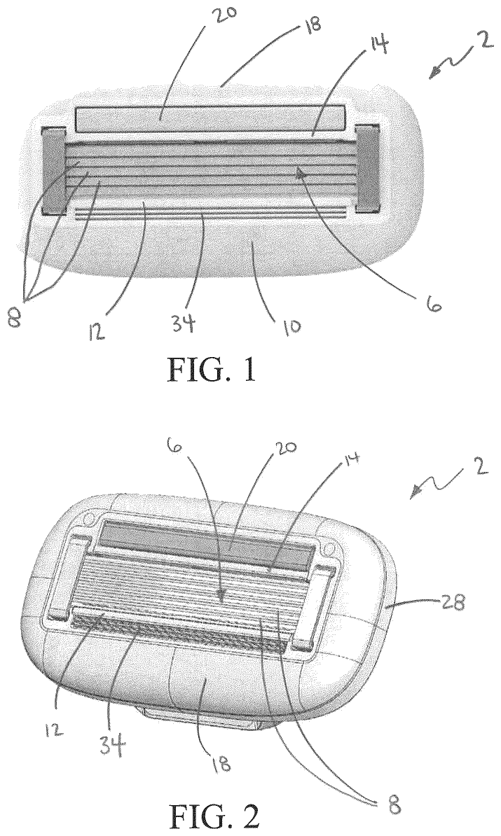

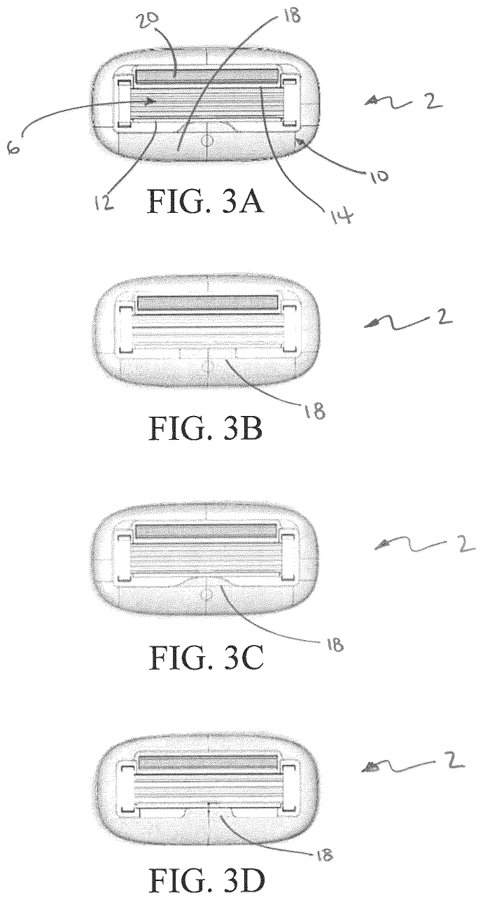

As shown in the attached drawings, including FIGS. 1-2 and 4, the present claimed invention is directed to a razor cartridge 2 comprising a core 4 including a blade mounting portion 6 configured to receive one or more blades 8, a frame 10 surrounding the blade mounting portion 6, a guard 12 and a cap 14. The razor cartridge 2 includes skin contacting portions associated with one or more portions of the core 4, generally including the guard 12, cap 14 and the frame 10, as well as a lube strip 20. As best shown in FIGS. 3A-3D, 4, 7 and 11, one or more of the skin contacting portions comprise a lubricious layer 18 formed of a lubricious layer composition including a matrix component and a lubricious component.

The preferred material for forming the core 4 of the razor cartridge 2 is an acrylonitrile butadiene styrene (ABS), as is widely used in the art. However, other suitable materials may be used to form the core 4 or portions thereof.

The core 4 is shown in the attached figures to be unitarily formed to include the blade mounting portion 6, the guard 12, the cap 14 and the frame 10 surrounding the blade mounting portion 6 as a single molded part. However, it is understood that one or more of the elements making up the core 4 may be formed separately and attached to one another. For example, the blade mounting portion 6 and frame 10 can be independent members that are then attached to form the core 4. Similarly, the guard 12 and/or cap 14 can be formed of independent members and then attached to the blade mounting portion 6 or the frame 10 to form the core 4.

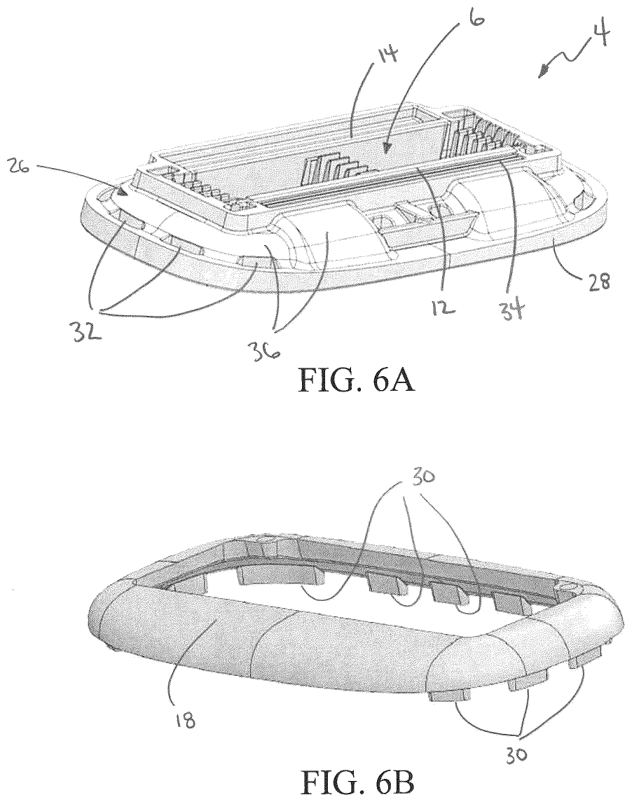

In a preferred embodiment shown in FIG. 6A, the core 4 comprises a blade mounting portion 6 integrally formed as one piece with a surrounding frame 10, where the lubricious layer 18 may be formed on the frame 10 surrounding the blade portion 6. This preferred embodiment provides a core 4 that eliminates the gap between the blade portion 6 and the frame 10 when the frame 10 is formed separately and attached to the blade portion 6. Reducing or eliminating the space between the blade portion 6 and the frame 10 provides an additional benefit in maintaining the lubricious layer 18 in the closest proximity to the blades 8, placing more lubricating material directly into contact with the skin adjacent the blades 8.

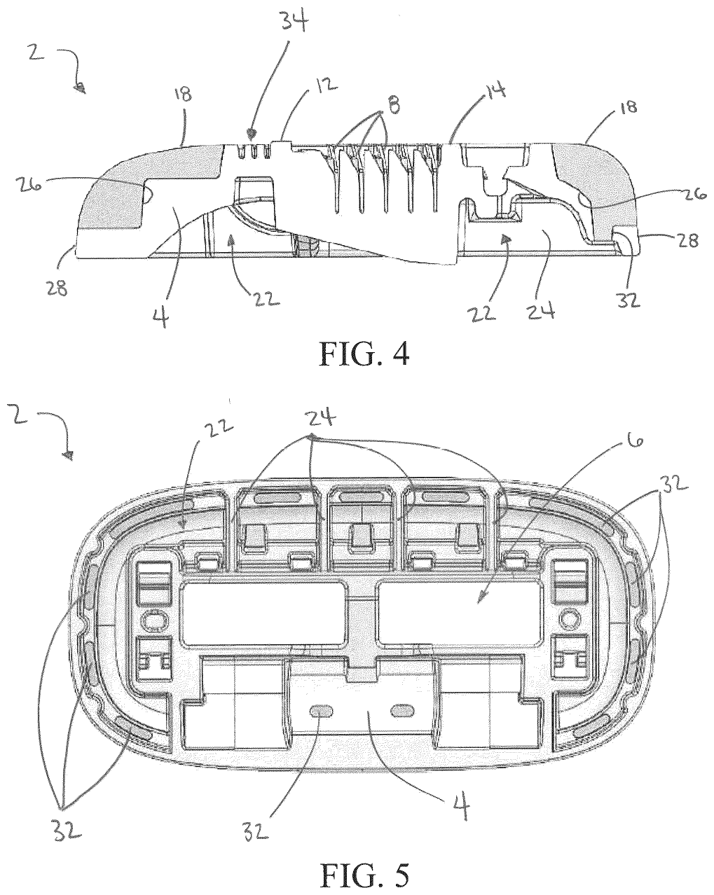

As shown in FIGS. 4 and 5, the preferred core 4 has a concave rear surface 22 for reducing the amount of material used to form the core 4. The concave rear surface 22 is preferably provided with support members 24, such as the ribs shown in the attached drawings, to provide additional integrity to the concave rear surface 22. In this regard, the support members 24 are especially placed in the areas of the concave rear surface 22 corresponding to the application of the lubricious layer 18 during overmolding of the lubricious layer composition to provide additional core integrity, helping to reduce warpage of the shave plane during manufacture of the razor cartridge 2. This is particularly important when a hot, melted lubricious layer composition material is injection molded onto a plastic core 4 that has cooled.

The preferred core 4 also comprises cut-away areas 26 for receiving the lubricious layer composition when forming the lubricious layer 18 on the core 4. See FIGS. 4, 6A, 7 and 11. In a preferred embodiment, cut away portions 26 terminate in a collar 28 that runs around at least a portion, if not the entirety, of a bottom edge of the core 4, to provide the smooth transition from the lubricious layer 18 to the core 4. See FIGS. 2, 4, 6A, 7, and 13. This provides the preferred razor cartridge 2 comprising a lubricious layer 18 on the frame 10 of the core 4 with an overall uniform surface absent edges or steps at the areas of the lubricious layer 18.

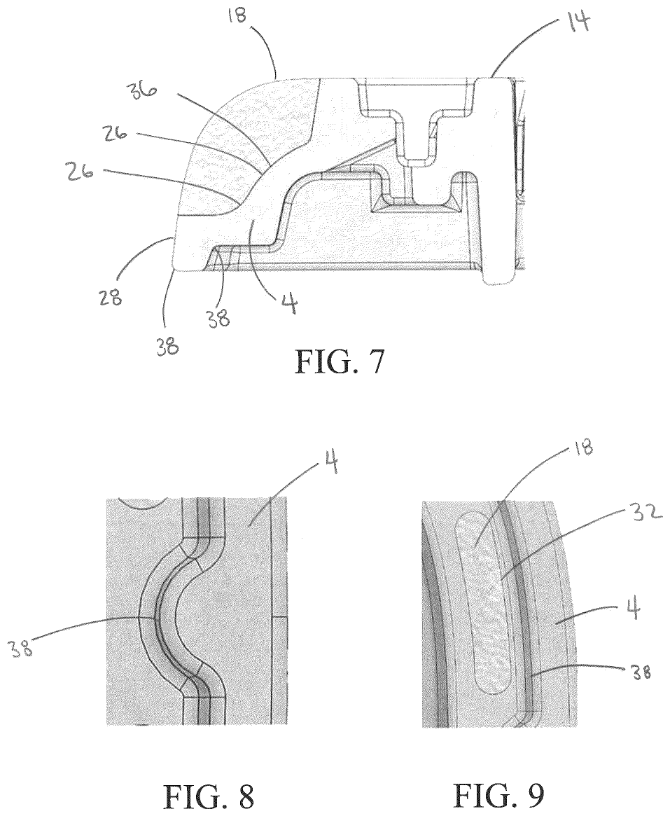

In the preferred embodiment shown in FIGS. 1, 3A-3D, 4, 5, and 7, the lubricious layer 18 is overmolded directly onto the core 4 in areas of the frame 10 associated with skin contacting portions of the razor cartridge 2. The preferred core 4 comprises rounded core surfaces 36, shown in FIGS. 6A and 7, that aid in the melt flow of the lubricious layer material onto the core 4. In a most preferred embodiment, the lubricious layer material is overmolded directly onto the core 4, rather than extruding pellets to strips and then extruding strips into the final shape. The single processing reduces breakage of lubricant long chain molecules, common with the heat and shear encountered using double-extrusion processing. This results in more lubrication per unit volume of the lubricious layer material.

A preferred embodiment related to a comolded lubricious layer 18 further comprises interlocking features 30 between the core 4 and the lubricious layer 18 to improve the physical connection between the core 4 and the lubricious layer 18. The use of such interlocking features 30 also help to reduce warpage of the shave plane of the razor cartridge 2 caused by comolding, thereby maintaining a more precise shave plane.

Preferred interlocking features 30 may include openings 32 that pass through the core 4, from the front surface to the back surface of the core 4, as shown in FIGS. 4, 5, 6A, 6B, and 9. In such an embodiment, interlocking features 30 formed of the lubricious layer composition fill the openings 32 when injection molded onto the core 4. Interlocking features 30 of varying length aids in the flow of the lubricious layer composition material along the varying curvature of the surface perimeter of the core 4, with the interlocking features 30 preferably located on flat surfaces to improve ejection of the part from the mold tool.

Rounded surfaces on the perimeter surfaces 38 of the core 4 comprising a change of direction, as well as an exaggerated draft on the collar 28 also improve ejection of the part. See FIGS. 7, 8 and 9. For example, a seven-degree exaggerated draft on the collar 28 of the core 4 may be preferred to reduce warpage due to possible core material or lubricious layer composition material sticking to the mold tool. Extra core material on outside surfaces of the core 4, such as the collar 28, minimizes the amount of the lubricious layer composition material used while maximizing the surface area of the lubrication layer 18 at the skin contacting portions of the razor cartridge 2.

The lubricious layer 18 can also be independently formed and attached to a molded razor cartridge 2 as part of a skin contacting portion of the razor cartridge 2, such as the attachment of an independently formed lube strip 20 in a designated area on the core 4. Attachment of a lubrication layer 18 as the lube strip 20 can be either instead of or in addition to a lubrication layer 18 applied to any other skin contacting portions of the razor cartridge 2.

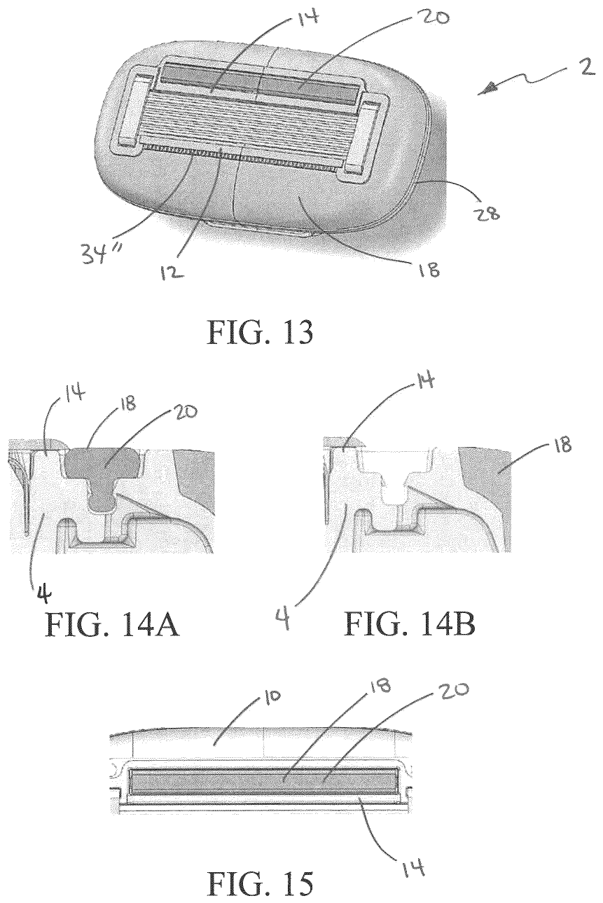

For example, as shown in FIGS. 13, 14A, 14B and 15, the core 4 can have an area in the vicinity of the cap 14, and preferably above the cap 14 as is well known in the art, where the lube strip 20 is attached to the core 4. The lube strip 20 shown in FIG. 14A, comprising a lubricious layer 18, is affixed to the core 4 by snapping the lube strip 20 into an opening in the core 4, as shown in FIG. 14B. The lube strip 20 can also be affixed to core 4 by an adhesive, by heat welding, by overmolding the lubricious layer 18 onto the core 4 in a designated area, and/or by other means available to affix the lubricious layer 18 to the core 4.

In a most preferred embodiment, the cartridge 2 comprises a first lubricious layer 18 on at least a portion of the frame 10 surrounding at least a portion of the blade portion 6, and a second lubricious layer 18 in the form of a lube strip 20, preferably in the area of the cap 14. As shown in FIG. 1, among others, the lube strip 20 is formed on the blade portion 6 of the core 4, separate from the lubricious layer 18 formed on the frame 10.

The separate lubricious layer 18 on at least a portion of the frame 10 and the lube strip 20 in the area of the blade portion 6 allow for the use of lubricious layer materials having different physical characteristics, including such characteristics as material composition, solubility and viscosity. For example, the lube strip 20 attached to the core 4 in the area of the cap 14 may have more lubrication than the lubricious layer 18 on the frame 10.

Although the guard 12 of the razor cartridge 2 can be formed of an independently formed thermoplastic elastomer fin structure, as is well known in the art, a preferred embodiment of the present invention comprises a rigid guard 12 being formed of the core material, i.e., ABS, as an integral part of the core 4 when injection molded. Such a rigid plastic guard 12 is preferably a one piece structure extending across virtually the length of the blades 8.

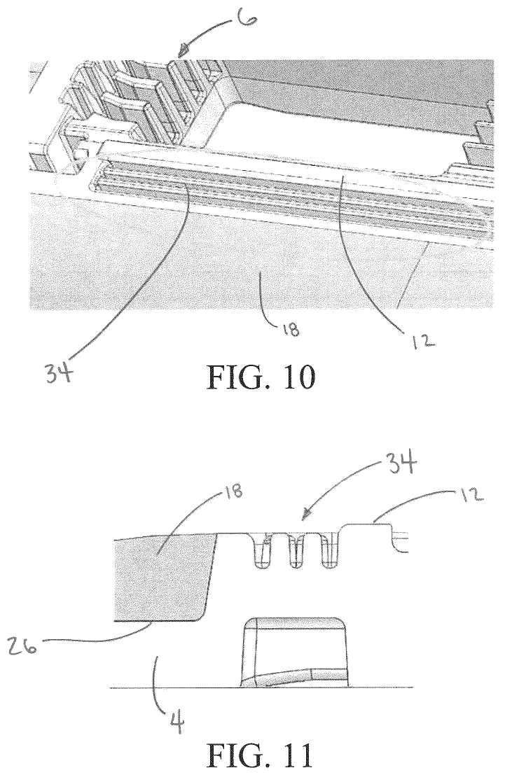

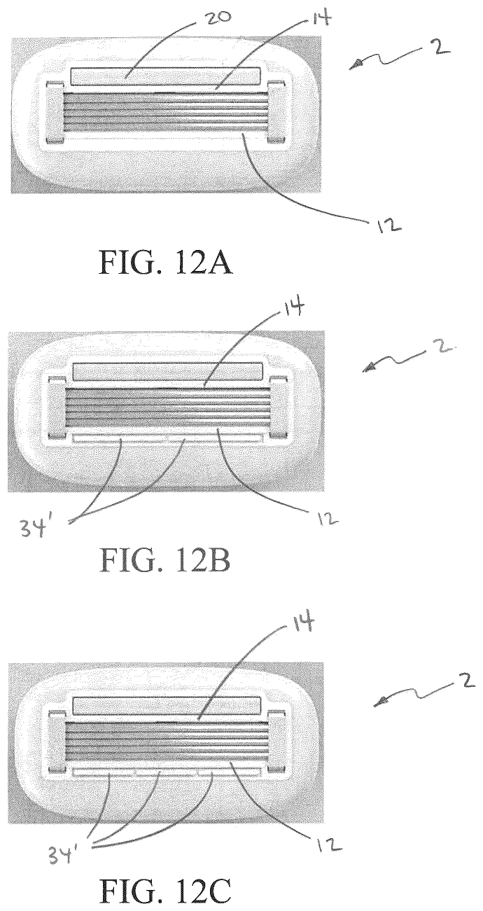

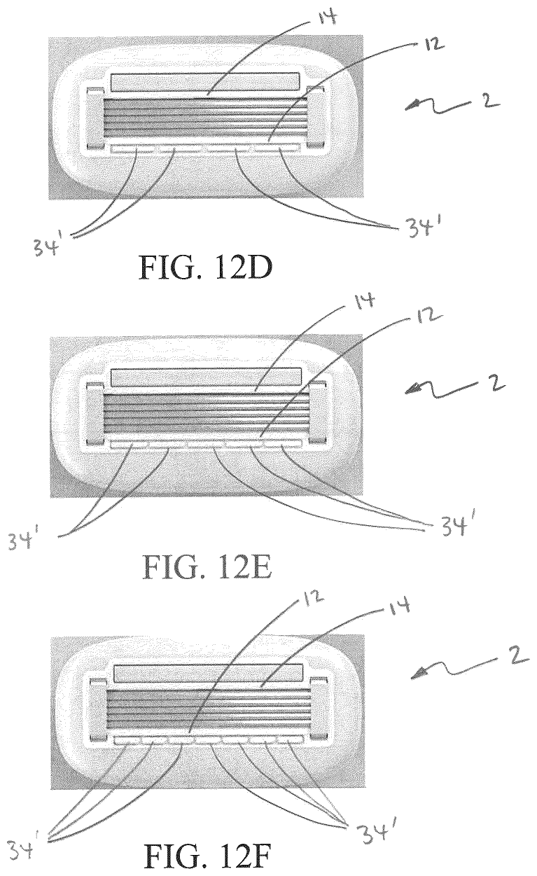

As shown in FIGS. 1, 2, 4, 10, 11 and 12A-12F, the guard 12 may be either in the form of, or used in connection with, guard fins 34 placed in front of a guard 12. Such guard fins 34 may be formed as a single or multiple continuous rigid members running parallel to the blades 8. Alternatively, as shown in FIGS. 12A-12F, one or more guard fins 34 may comprise two or more segments 34′ across a longitudinal length, with breaks between longitudinal segments 34′ of the guard fins 34. In another embodiment, shown in FIG. 13, the fin guard 34 may be in the form of a comb guard 34″, having alternating segments and breaks.

The matrix component of the lubricious layer composition preferably comprises a biodegradable copolymer, and most preferably polybutylene adipate terephthalate (PBAT), with reduced polystyrene content and composition components that enhance the use of the materials in a lubricious layer 18 on the razor cartridge 2.

The most preferred formulation of the lubricious layer composition comprises about 67.84 weight percent polyethylene glycol 115M (PEG115M); about 20 weight percent polystyrene; about 7.8 weight percent polybutylene adipate terephthalate (PBAT); about 3.99 weight percent polyethylene glycol 90 (PEG90); about 0.1 weight percent tocopherol; about 0.25 weight percent of a dye; and about 0.02 weight percent of aloe vera as a care agent.

When formulating the lubricious layer composition, it is important to provide a thorough grinding of the PBAT component prior to compounding the components, preferably by cryogenic grinding. In a most preferred embodiment, the PBAT is uniformly ground to from about 0.1 to about 5.0 micrometers when used in the present teaching.

The PBAT can be ground beforehand and formulated with the other components, so that all of the components, including the PBAT, are compounded together to obtain a granulate ready lubricious layer composition for injection molding. In another embodiment the ground PBAT can be added to the remainder of the mixed lubricious layer composition components directly before injection molding.

For example, the finely ground PBAT can be mixed with the other lubricious layer composition components prior to extrusion, followed by extruding directly with the previously ground PBAT, where the extrudate is hot-die-granulated to obtain a granulate suitable for injection molding. Alternatively, the lubricious layer composition components other than the PBAT can be extruded and granulated, i.e., using hot die granulation, only then adding the PBAT as a granulate during the injection molding process.

Variations, modifications and alterations to the above detailed description will be apparent to those skilled in the art. All such variations, modifications and/or alternatives are intended to fall within the scope of the present invention, limited only by the claims. Any cited patents and/or publications are incorporated by reference.

We claim:

1. A razor cartridge comprising a core including a blade mounting portion configured to receive one or more blades and at least one of a portion of a frame, a guard and a cap, said razor cartridge comprising one or more skin contacting portions associated with one or more portions of the core, wherein one or more of the skin contacting portions comprise a lubricious layer formed of a lubricious layer composition including a matrix component and a lubricious component.

2. The razor cartridge of claim 1 wherein the lubricious layer is formed directly onto at least a portion of the core by injection molding the lubricious layer composition onto the core.

3. The razor cartridge of claim 1 wherein the lubricious layer is formed independently from the core and then subsequently attached to the core.

4. The razor cartridge of claim 1 wherein at least a portion of the core has a concave rear surface.

5. The razor cartridge of claim 4 wherein the concave rear surface comprises one or more supports.

6. The razor cartridge of claim 1 wherein the core comprises one or more cut-away areas in which the lubricious layer is fashioned.

7. The razor cartridge of claim 6 wherein at least a portion of the cut-away areas terminate in a collar extending around at least a portion of a bottom edge of the core.

8. The razor cartridge of claim 1 wherein the matrix component of the lubricious layer composition comprises a PBAT.

9. The razor cartridge of claim 1 wherein PBAT comprises from about 2 to about 20 weight percent of the lubricious layer composition.

10. The razor cartridge of claim 1 wherein PBAT comprises from about 5 to about 15 weight percent of the lubricious layer composition.

11. The razor cartridge of claim 1 wherein the blade mounting portion and at least a portion of the frame of the core on which a lubricious layer is formed are integrally formed.

12. The razor cartridge of claim 1 comprising more than one lubricious layer, including a first lubricious layer that is overmolded onto the core and a second lubricious layer that is formed independently from the core and attached to the core.

13. The razor cartridge of claim 12 wherein the first lubricious layer is molded directly onto at least a portion of the core.

14. The razor cartridge of claim 12 wherein the first lubricious layer and the second lubricious layer have different lubrication characteristics.

15. The razor cartridge of claim 12 wherein the first lubricious layer and the second lubricious layer are formed of different compositions.

16. The razor cartridge of claim 12 wherein the first lubricious layer and the second lubricious layer are located independent of one another, on different parts of the core.

17. A method of manufacturing a razor cartridge comprising:
a. integrally forming a core comprising a blade mounting portion configured to receive one or more blades and at least a portion of a frame;
b. molding a lubricious layer formed of a lubricious layer composition including a matrix component and a lubricious component onto at least a portion of the frame; and
c. attaching a lube strip to the core.

18. The method of claim 17 wherein the distance between one of the one or more blades and the lubricious layer is minimized.

19. The method of claim 17 wherein the step of molding the lubricious layer onto at least a portion of the frame is an initial processing of the lubricious layer composition.

20. The method of claim 17 wherein the lubricious layer and the lube strip have different physical characteristics.
